(19) United States
(12) Patent Application Publication
(10) Pub. No.: US 2006/0210436 A1
(43) Pub. Date: Sep. 21, 2006

(54) NEMATIC LIQUID CRYSTAL THIN FILMS FOR CHEMICAL VAPOR SENSING

(75) Inventor: Devanand K Shenoy, McLean, VA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignee: THE GOVERNMENT OF THE UNITED STATES OF AMERICA, AS REPRESENTED BY THE SECRETARY OF THE NAVY, Washington, DC (US)

(21) Appl. No.: 11/307,965

(22) Filed: Mar. 1, 2006

Related U.S. Application Data
(60) Provisional application No. 60/662,341, filed on Mar. 15, 2005.
(60) Provisional application No. 60/662,340, filed on Mar. 15, 2005.

(51) Int. Cl. G01N 33/00

(52) U.S. Cl. 422/68.1; 422/82.05

(57) ABSTRACT

A device for detecting an analyte having a substrate, an alignment layer on the substrate, a film having 4-pentyl-4′-cyanobiphenyl on the alignment layer, a flow cell capable of delivering air suspected of containing the analyte to the film, and an apparatus capable of measuring a physical property of the film. A method of detecting an analyte by: providing a device having a substrate, an alignment layer on the substrate, and a film having 4-pentyl-4′-cyanobiphenyl on the alignment layer; exposing the film to air suspected of containing the analyte; and measuring a change in a physical property of the film in response to exposing the film.

### CROSS REFERENCE TO RELATED APPLICATIONS

This application claims priority to U.S. Provisional Patent Application Nos. 60/662,340 and 60/662,341, both filed on Mar. 15, 2005, and both incorporated herein by reference. US Patent Application to Shenoy et al. entitled “CAVITANDS FOR CHEMICAL VAPOR SENSING,” designated as 97183US2, and filed on the same day as the present application, is incorporated herein by reference.

### FIELD OF THE INVENTION

The invention relates generally to chemical vapor sensing

### DESCRIPTION OF RELATED ART

Environmental pollution due to hazardous chemical vapors is an issue of critical importance due to its adverse effects on health and global warming. Chemical detectors to sense the presence of such organic compounds as aromatic and halogenated hydrocarbons are available¹⁻⁹ based on a variety of sensing layers and the development of multi-pixel arrays.¹⁰⁻¹³ A majority of these sensors are based on polymer coatings as sensing layers.^{1-3,5-9,11,12} Recently, supramolecular materials have also been demonstrated as suitable coatings for sensors.¹⁴⁻²¹ The interaction of chemical vapors with these sensing layers is transduced using, for example, mass transduction techniques such as Quartz Crystal Microbalance (QCM),^{6,18} Surface Acoustic Wave (SAW)^{7,19} or optical methods such as Surface Plasmon Resonance (SPR).^{16,20,21} Electrical transduction schemes using capacitance measurements on interdigitated electrodes have also been reported.²²

Liquid crystals (LC) have amplifying properties.²³⁻³⁰ This amplification is a result of the cooperative realignment of liquid crystal molecules by external perturbations, in this case the presence of a chemical vapor.³¹⁻³⁴ Cholesteric and nematic liquid crystal mixtures have been used as sensitive layers on mass sensitive transducers.²⁷ The variation in partition coefficient for different chemical vapors gives rise to sensor response which differs for each type of chemical, suggesting that LC sensing layers may be used to produce a sensor with some selectivity.²³⁻²⁸ These studies have been conducted with liquid crystals that are not uniformly aligned, therefore selectivity and sensitivity have been comparable to that achieved using isotropic polymer sensing layers. Recently, it has been shown that multi-component mixtures of homogenously aligned nematic liquid crystals can be used as sensing layers for volatile organic compounds using optical transducers and inter-digitated capacitor structures.^{29,30}

### BRIEF SUMMARY OF THE INVENTION

The device for detecting an analyte comprises a substrate, an alignment layer on the substrate, a film comprising 4-pentyl-4′-cyanobiphenyl on the alignment layer, a flow cell capable of delivering air suspected of containing the analyte to the film, and an apparatus capable of measuring a physical property of the film.

The method of detecting an analyte comprises: providing a device comprising a substrate, an alignment layer on the substrate, and a film comprising 4-pentyl-4′-cyanobiphenyl on the alignment layer; exposing the film to air suspected of containing the analyte; and measuring a change in a physical property of the film in response to exposing the film.

### BRIEF DESCRIPTION OF THE DRAWINGS

A more complete appreciation of the invention will be readily obtained by reference to the following Description of the Example Embodiments and the accompanying drawings.

FIG. 1 schematically illustrates an SPR sensing apparatus.

FIG. 2 shows a typical SPR time response curve for benzene in contact with 5CB at room temperature as a function of the applied concentration (60, 75, 115, 150, 230, 370, 580, 1670, 2340, and 5840 ppm respectively from time 0 to 60 min.). The inset graph shows the SPR time response curve for benzene at low concentration starting from 60 ppm up to 230 ppm.

FIG. 3 shows time dependence of the plasmon resonance wavelength on exposure to high concentration of benzene vapors of nematic liquid crystal at room temperature (1 and 2 are concentration at 11700 ppm).

FIG. 4 shows calibration curves of the shift of resonance wavelength versus concentration for 5CB nematic liquid crystal at room temperature exposed to different analyte vapors at very low concentration (benzene (□), m-xylene (Δ), toluene (▪), p-xylene (▴), acetonitrile (◯), and ethyl acetate (●)).

FIG. 5 shows selectivity patterns of 5CB nematic liquid crystal towards analyte vapors at 110 ppm.

FIG. 6 shows selectivity patterns of 5CB nematic liquid crystal towards analyte vapors at relative vapor pressure 0.0012.

FIG. 7 shows time dependence of plasmon resonance wavelength for benzene at a relative saturation pressure 0.0012 in contact with 5CB at room temperature (the solid line represent the best fit of the experimental data, assuming second order adsorption kinetics).

FIG. 8 shows results of the same test as FIG. 7 using m-xylene.

FIG. 9 shows time dependence of the plasmon resonance wavelength on exposure to different concentration of m-xylene vapors of nematic liquid crystal close to nematic-isotropic phase transition temperature: 1) 0.27 ppm, 2) 0.38 ppm, 3) 0.68 ppm, 4) 0.85 ppm.

FIG. 10 shows time dependence of plasmon resonance wavelength for m-xylene at 0.38 ppm in contact with 5CB close to nematic-isotropic temperature.

### DETAILED DESCRIPTION OF EXAMPLE EMBODIMENTS

In the following description, for purposes of explanation and not limitation, specific details are set forth in order to provide a thorough understanding of the present invention. However, it will be apparent to one skilled in the art that the present invention may be practiced in other embodiments that depart from these specific details. In other instances, detailed descriptions of well-known methods and devices are omitted so as to not obscure the description of the present invention with unnecessary detail.

Described is a method to use thin films of 4-pentyl-4′-cyanobiphenyl (5CB) liquid crystal that can be spin coated onto surfaces. The purpose of this is to enhance the selectivity of detection for chemical vapors. The 5CB may cooperatively amplify the perturbation caused by exposure to chemical vapors so as to allow for a highly sensitive chemical vapor detector.

A problem with current chemical vapor detectors is that they are still prone to false alarms and are relatively bulky. The problem of false alarms is caused by the limited selectivity and sensitivity of sensing layers for chemical threats and therefore there is a need for new materials with enhanced selectivity and sensitivity. The widely available nematic liquid crystal 5CB may be used as a sensing layer for chemical vapors such as toluene, benzene, m-xylene, p-xylene, acetonitrile, and ethyl acetate. 5CB has a convenient room temperature nematic range and high chemical stability even in the presence of water vapor and oxygen.³⁵ This nematic liquid crystal also has a large positive refractive index anisotropy (on the order of 0.2) rendering it suitable for significant sensitivity amplification due to a phase transition into the isotropic phase. Once the LC layer is deposited on a gold-coated optical substrate, the white-light SPR transduction method allows for efficient measurement of the refractive index (RI) change induced in the LC layer by exposure to chemical vapors.

The surface plasmon resonance technique is an effective transduction scheme for probing the change in single component liquid crystal order due to chemical vapor exposure. The liquid crystal order may be perturbed to different extents by different vapors, which can lead to selectivity. At high concentrations of the vapor, a complete phase transition from the nematic to the isotropic phase may not be induced, as confirmed by polarized optical micrography, although the SPR response may show signal saturation. Depending on the structure and shape of the vapor molecule, the transition from ordered nematic to disordered nematic may take place at different relative concentrations of vapor. Close to the nematic-isotropic phase transition temperature the sensitivity can be enhanced and therefore used to detect concentrations of analyte at low ppm levels. The kinetics of response to different vapors may be different. This could be considered an additional parameter for determining selectivity. Further, due to the weak intermolecular forces between chemical vapor and LC, recycling of the sensor may be feasible.

The device may enable highly sensitive and selective chemical vapor detectors that rely on coating materials. Applicability may be broad in that the liquid crystal material can be coated onto many different transduction platforms: optical platforms such as surface plasmon resonance, optical waveguides, interferometers; electrical platforms such as capacitive transducers; mechanical platforms such as AFM cantilevers, SAW devices and QCM microbalances that translate the mass change from analyte deposition onto liquid crystal into a frequency change that can be measured.

The high selectivity is seen from the ability to discriminate between chemical isomers such m-xylene and p-xylene. It is also seen that holding the temperature of the 5CB closer to a phase transition (about 5° C. below the nematic-isotropic phase transition) temperature may increase the sensitivity of the 5CB by two orders of magnitude. This sensitivity may be increased even further by holding the liquid crystal closer to the phase transition temperature (for example, 0.1° C. below the phase transition). However, if a large dynamic range is desired instead of sensitivity, the liquid crystal may be held further away from the phase transition.

Uniformly aligned nematic liquid crystal films may be obtained by spin coating the liquid crystal layer onto a rubbed polyimide surface. This eliminates the need for a second alignment layer to be used (which is typically the case for most liquid crystal applications i.e. the liquid crystal is sandwiched between two plates whose inner surfaces have an alignment layer).

The liquid crystal film may also comprise a cavitand molecule, such as, but not limited to, those disclosed in the US Patent Application to Shenoy et al. entitled “CAVITANDS FOR CHEMICAL VAPOR SENSING,” designated as 97183US2, and filed on the same day as the present application

The kinetics of the SPR response may be used as an additional selectivity parameter in addition to the strength of the SPR signal. This may allow for further discrimination between chemical vapors.

The substrate can by made from any material that is compatible with the alignment layer and with the method of measuring the physical property. When the physical property is optical or is measured by optical techniques, the substrate may be a transparent material, such as but not limited to glass. Additional layers may be coated on the glass such as a chromium adhesion layer and a gold layer. The alignment layer can assist in making the 5CB layer isotropic. Suitable alignment layers include, but are not limited to, rubbed polyimide.

A variety of physical properties of the film may be measured, such as, but not limited to, optical, refractive index, electrical, and mass. Suitable apparatus and/or methods for measuring the physical property include, but are not limited to, surface plasmon resonance, interferometry, capacitive transductance, atomic force microscope, surface acoustic wave, and quartz crystal microbalance.

The device may include a system, such as but not limited to a computer, for correlating a change in the physical property of the film and/or the kinetics of such a change to information regarding the analyte, such as but not limited to, concentration and identification. The device may also include a temperature controller. This may be used to maintain the temperature of the 5CB at a temperature that may enhance sensitivity. Such temperature control may occur during the exposing, the measuring, or both.

Having described the invention, the following examples are given to illustrate specific applications of the invention. These specific examples are not intended to limit the scope of the invention described in this application.

### EXAMPLE 1

Fabrication of substrate with 5CB—To prepare substrates for surface plasmon resonance measurements, an approximately 2 nm thick chromium adhesion layer was first deposited onto a cleaned glass substrate (1 mm×20 mm×20 mm, n_{D}=1.92286 SNPH2, Optimax System Inc., USA) using a vacuum evaporator (Edwards Auto 306). This was followed by vapor deposition of a nearly 50 nm thick gold layer using a gold coin (Canadian coin, 99.99%). Evaporation was performed at a vacuum of 10⁻⁶ bar. The evaporation rate, as indicated by the read out on the instrument, was in the range of 0.02-0.04 nm/s. The thickness of the deposited metal film was determined by a quartz crystal thickness monitor.

Next a rubbed polyimide alignment layer was fabricated on top of the gold layer. For this, a solution of 1:100 (w/w) polyimide (PI2556):solvent (T9039) was spin coated onto the gold-coated substrate at 6000 rpm for 40 sec. The substrate was heated on a hot plate at 80° C. for 3 min to let the solvent evaporate and then heated further for 2 hr at 250° C. in an oven. The thickness of the alignment layer was determined to be between 10 to 15 nm by multi-wavelength spectroscopic ellipsometry (EC110) at an incident angle of 70 degrees. The unidirectional rubbing of the polyimide alignment layer was performed using a commercial automated rubbing machine (SPB8OPN, SUPER PILLOW BLOCK, THOMSON INDUSTRIES INC.).

Homogenously aligned 5CB films with a free air interface were then deposited as follows.³⁶ A flat Mylar O-ring, ˜100 μm thick and ˜5 mm inner diameter was placed on the substrate. A small drop of 5CB (˜1 microliter) in the isotropic phase (the liquid crystal was heated until it was clear indicating that it is above 35° C., the nematic to isotropic phase transition temperature of 5CB) was dispensed from a hypodermic needle into the well formed by the Mylar O-ring. The droplet completely wet and spread uniformly over the polyimide alignment layer, but was contained by the O-ring, as 5CB does not wet a mylar surface. The substrate was then slowly cooled to room temperature. The optical textures were observed using polarized optical microscopy to be characteristic of uniformly aligned nematic films. The thickness of the liquid crystal material deposited was controlled by the amount of 5CB dispensed into the well and the size of the well.

### EXAMPLE 2

SPR apparatus—Real-time measurement of the refractive index (RI) of the sensing layers was performed using a custom-built white-light SPR instrument. White-light SPR is a well-established technique for measuring changes in the refractive index of thin layers.^{30,37} In the Kretschmann configuration³⁸ used in this white-light SPR sensor, TM-polarized, collimated white light (wavelength range ˜500-1100 nm) directed through the side of a high-RI prism strikes the gold-coated sensing surface at an angle above the critical angle. The light reflected from this surface is collected and analyzed by a spectrophotometer. Certain wavelengths of incident light will excite surface plasma waves at the interface between the gold layer and the sensing layer, and the loss of this energy will be observed as a decrease in reflectivity at those wavelengths. The wavelengths at which this occurs vary with the refractive index of the sensing layer near the gold surface, therefore a measurement of RI may be obtained by analysis of reflection spectra.

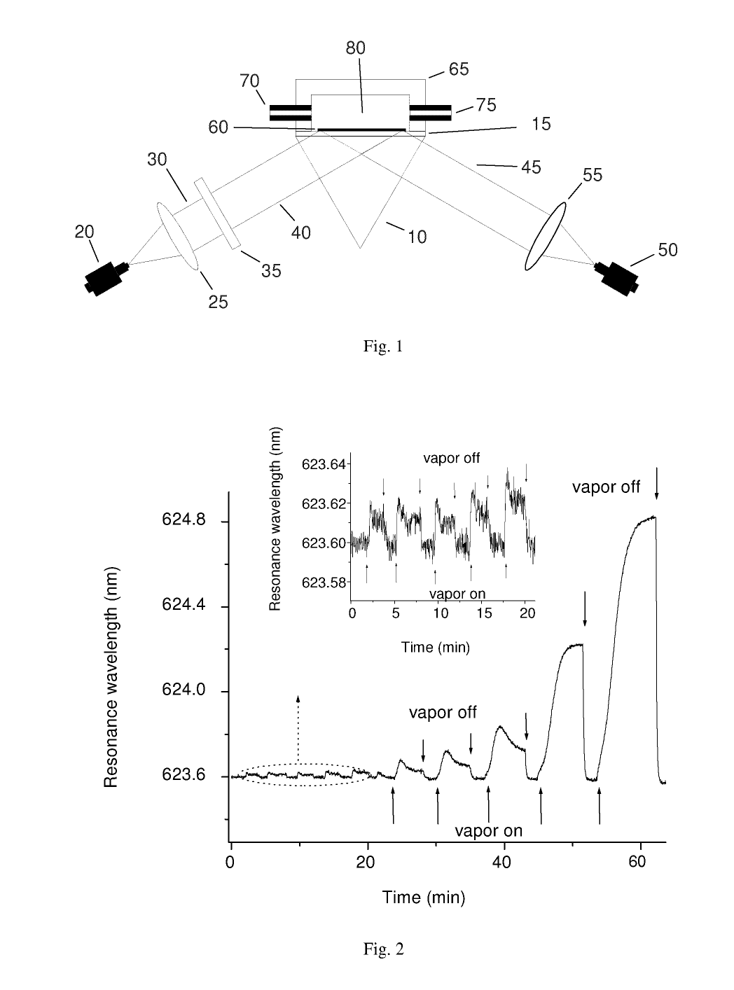

The instrument is similar to that described by Homola et al.³⁷ but modified for ease of adjustment and operation at high RI. A schematic of the instrument is shown in FIG. 1. Rather than depositing layers directly on the prism 10, the flat polyimide coated substrate 15 with 5CB layer 60 described above is index matched to a prism (equilateral, basis 20 mm×20 mm, n_{D}=1.92286 SNPH2, Optimax System Inc., US) using Cargille fluid (n_{D}=1.92). A multimode fiber optic 20 illuminated by a halogen fiber optic illuminator (5v/5 w halogen bulb, Ocean Optics) placed at the focus of a convex achromatic lens 25 is used to generated collimated polychromatic light 30. A rotatable polarizer 35 placed after the collimating lens allows either the TM-polarized component to be selected (for SPR sensing) or the TE-polarized component (to provide an intensity reference that is not affected by SPR). The polarized light 40 is directed onto the entrance face of the prism 10 at an adjustable angle (68 degrees nominal). Following reflection from the sensing surface, the light exits the opposite face of the prism 45 and is focused onto another multimode fiber 50 by a second convex achromat 55. The fiber carries the light to a spectrometer (Ocean Optics USB2000), where it is analyzed. As the surface plasmon wave is only excited by the TM-mode, the TE-mode intensity is used as a reference signal.

To allow control of the exposure of the substrate to organic vapors, a Teflon flow cell 65 comprising an inlet 70 and outlet 75 for the chemical vapors and a rectangular aperture 80 for the prism and substrate was constructed. An O-ring ensured a good seal between the prism/substrate and Teflon chamber. A temperature controlled water circulating unit (control to within 0.1° C.) was coupled to the Teflon chamber through a metal coil to control the liquid crystal temperature.

To generate ppm levels of organic vapors, a diffusion vial (D-5.0 mm capillary, VICI Metronics), filled with the organic compound using 5 mL syringe needles (VICI Metronics), was placed in a U-tube containing glass beads on one side of the tube and diffusion vial on the other. The U-tube was placed in a temperature controlled water bath (controlled to within 0.1° C.) from PolySciences, Inc. The chemical vapors were diluted with a stream of dinitrogen (carrier gas). The flow of both the carrier gas and the chemical vapor were controlled to within an accuracy of 1-2% using computer-interfaced mass flow controllers (DFC26, AALBORG INC.) and mixed in the appropriate proportion before being introduced into the flow cell. Care was taken to ensure that the experiment was performed after the vapor had attained equilibrium with respect to its concentration.

Alignment of the 5CB liquid crystal film on the rubbed polyimide coated substrate was examined using transmission polarized light microscopy. As expected for an optically uniaxial film, rotation of the substrate caused the intensity of transmitted light to go from a maximum (when the liquid crystal optic axis is oriented at an angle of 45 degrees with respect to the axis of either polarizer or analyzer) to a minimum (when the optic axis is aligned parallel to the axis of either polarizer or analyzer). This implies that the alignment layer forces the LC molecules to preferentially align along the rubbing direction. Though the orientation at the air interface may be homeotropic, the uniformly dark and bright images confirm that the projection of the optic axis is oriented along the rubbing direction.³⁶

### EXAMPLE 3

Sensitivity to benzene—FIG. 2 shows the variation in SPR wavelength as the sensor is exposed to benzene vapors ranging in concentration from 60 ppm to 5840 ppm. The inset in FIG. 2 shows the surface plasmon resonance response to benzene concentrations ranging from 60 ppm to 230 ppm. The measurements at low concentrations had a characteristic initial sharp increase of the response, followed by a slight decay and stabilization after 0.5-1.0 min. For high concentrations, however, (>5840 ppm), the decay was not observed. Rather, the response stabilized immediately following the initial increase of response.

The shifts in response may be caused by the perturbation of liquid crystal due to incorporation of vapors and resulting decrease in the liquid crystal order. The height of the initial peak depended on the change of liquid crystal from ordered to less ordered due to vapor interaction with the LC. The subsequent decay of response at low concentrations may be explained as a result of a gradual change in order from disordered to somewhat more ordered as benzene molecules self-orient themselves within the liquid crystal molecules. As seen in FIG. 2, when the perturbed LC layer was exposed to nitrogen gas free of chemical vapors, the plasmon wavelength minimum rapidly shifted back to its value before vapor exposure. The chemical vapors typically interact with the liquid crystal through weak forces such as dipole-dipole interactions and dispersion forces and so this is not a surprising result. The reversible nature of the LC/vapor interaction suggests that LC layers can be used for repeated measurements during sensing applications (in FIG. 2, “vapor on” indicates the time where benzene vapor is turned on and “vapor off” indicate the time where stream of dinitrogen is turned on). It is noted that the chemical vapor remained within the liquid crystal bulk film until the nitrogen gas is introduced to remove the vapors.

### EXAMPLE 4

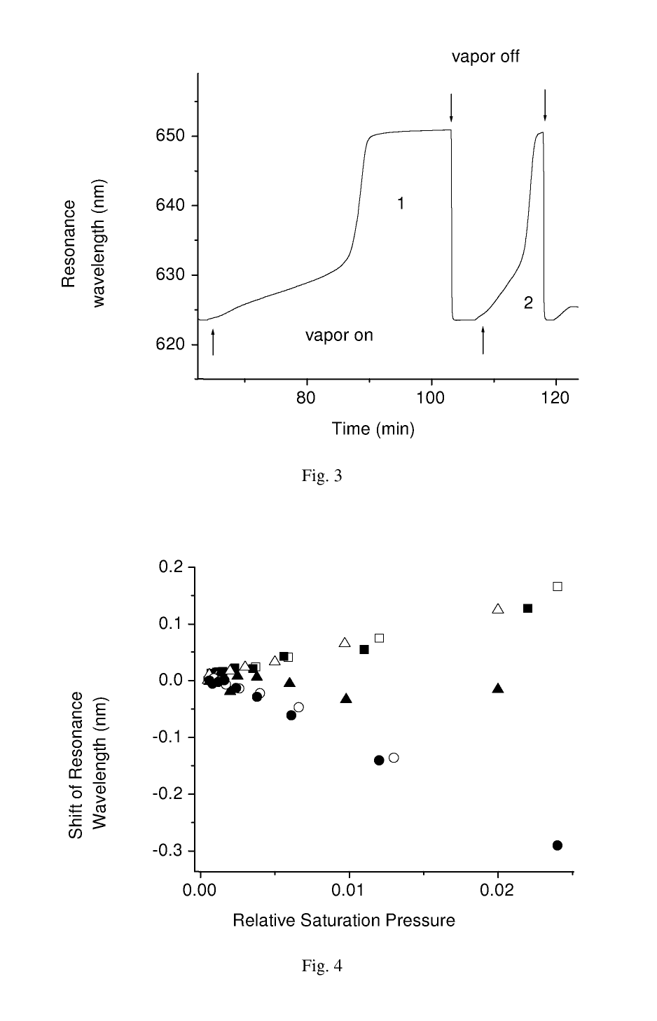

Upper limit of concentration measurement for benzene—FIG. 3 shows that for high concentrations of vapor (11690 ppm of benzene) a change was induced in the liquid crystal from the uniformly ordered nematic to a partially disordered nematic phase. The resonance wavelength shift from 622 nm to 652 nm (a shift of about 30 nm) was due to a significant perturbation of the liquid crystal. Once the liquid crystal is disordered at room temperature, further exposure to vapors or increase of concentration does not significantly change the degree of disorder. This represents an upper limit to the range of concentrations that can be observed and therefore defines the dynamic range at room temperature. Clearly therefore, since these measurements are performed well below the phase transition temperature of the LC (5CB undergoes a phase transition into the isotropic phase at 35.1° C.), the chemical vapors do not induce a phase change although they perturb the nematic order. The measurements shown in FIG. 3 have a characteristic slow increase of the response followed by a sharp increase and stabilization after 0.1 min. Similar response was observed when the liquid crystal was exposed to the other vapors at room temperature.

### EXAMPLE 5

Optical texture—The optical texture of 5CB after exposure to vapors was examined under a polarizing microscope and it was confirmed that the initial uniform alignment of the liquid crystal film could be recovered. The optical extinction was not observed at any orientation of liquid crystal substrate with respect to the polarizer. However, 5CB does not appear to undergo a clear phase transition into the isotropic phase (which would give a dark image at all orientation of the liquid crystal substrate). In contrast, when the regenerated liquid crystal substrate was rotated into different orientations with respect to polarizer, optical extinction was observed. These optical characteristics indicated that 5CB was oriented non-uniformly because of the presence of the vapors and oriented uniformly in the absence of the vapors.

### EXAMPLE 6

Other vapors—To demonstrate how the LC response is different for different types of chemical vapors, curves showing variation of resonance wavelength shift with respect to the relative saturation pressure of six different vapors are shown in FIG. 4. Four aromatics, benzene, toluene, m-xylene and p-xylene and two non-aromatic vapors, acetonitrile and ethyl acetate, were investigated. To test whether LC layers are potentially capable of distinguishing isomers, the aromatic isomers m-xylene and p-xylene were studied as well. It is seen that the calibration curves differ from each other and that the slope, which represents the sensitivity as seen in FIG. 4, is significantly different for each vapor (see Table below).

| | | | | Saturated |
|---|---|---|---|---|
| | | | Refractive | vapor |
| | | | index | pressure, |
| | | Linear | at 20° C. | at 20° C. |
| | Slope (nm/pₛ) | Regression | (n_{D}) | (mm Hg) |
| Benzene | 6.679 ± 0.214 | 0.99 | 1.4980 | 75 |
| Toluene | 5.396 ± 0.321 | 0.99 | 1.4960 | 20 |
| m-Xylene | 6.193 ± 0.176 | 0.99 | 1.4970 | 6 |
| p-Xylene | −1.241 ± 0.777 | 0.92 | 1.4950 | 3 |
| Acetonitrile | −9.788 ± 0.318 | 0.99 | 1.3440 | 73 |
| Ethyl acetate | −12.619 ± 0.259 | 0.99 | 1.3720 | 74 |

The resonance wavelength shift observed appears to be dependent upon both the refractive index of the vapor introduced and the extent of disorder induced (which can be different for different vapors). For example, both ethyl acetate and acetonitrile have lower refractive indices than the aromatic vapors, and so the resonance wavelength shift may be expected to be smaller when these vapors are partitioned into the liquid crystal film.

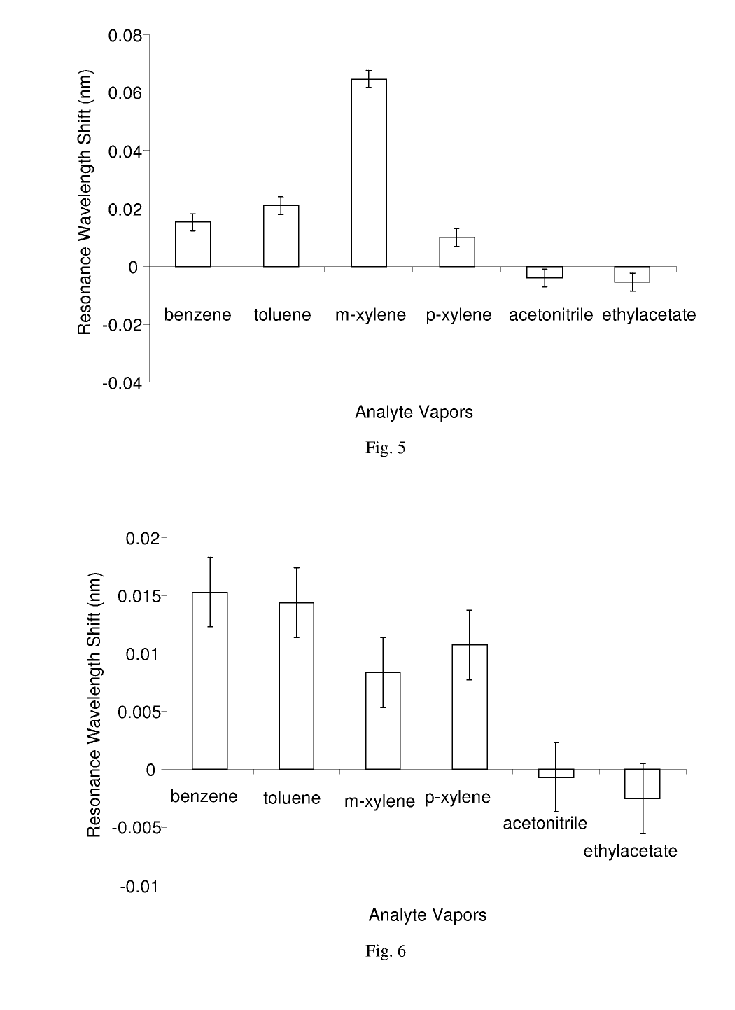

The bar graphs of FIGS. 5 and 6 give an overview of the difference in response of 5CB to all six chemical vapors at equal concentration (110 ppm, FIG. 5) and at equal relative vapor pressure (0.0012, FIG. 6) respectively. FIG. 5 clearly shows that since the mole fraction of chemical vapors is the same in all cases, the layer responds differently to different types of chemical vapor. However, when the resonance wavelength shift is compared at equal vapor pressures, the layer responds differently to different class of chemical vapors. FIG. 6 shows a clear selectivity pattern between aromatic and non-aromatic vapors. It could be assumed that the different shapes of the molecular structure of different chemicals used causes the changes in the signal response, but the situation is more complicated. This is because the resonance wavelength shift, as mentioned earlier, has contributions from both the different extents of disorder induced by different vapors and the specific refractive index of the vapors. A further complication in terms of analyzing the contributions to the refractive index change arises because SPR is sensitive only to changes caused by the TM wave and therefore to components of the refractive index normal to the surface. Also, the liquid crystal layer is not necessarily uniformly planar throughout the bulk. A small region at the air interface is typically homeotropic in orientation. There is therefore an effective refractive index that changes with the inclusion of vapors.

### EXAMPLE 7

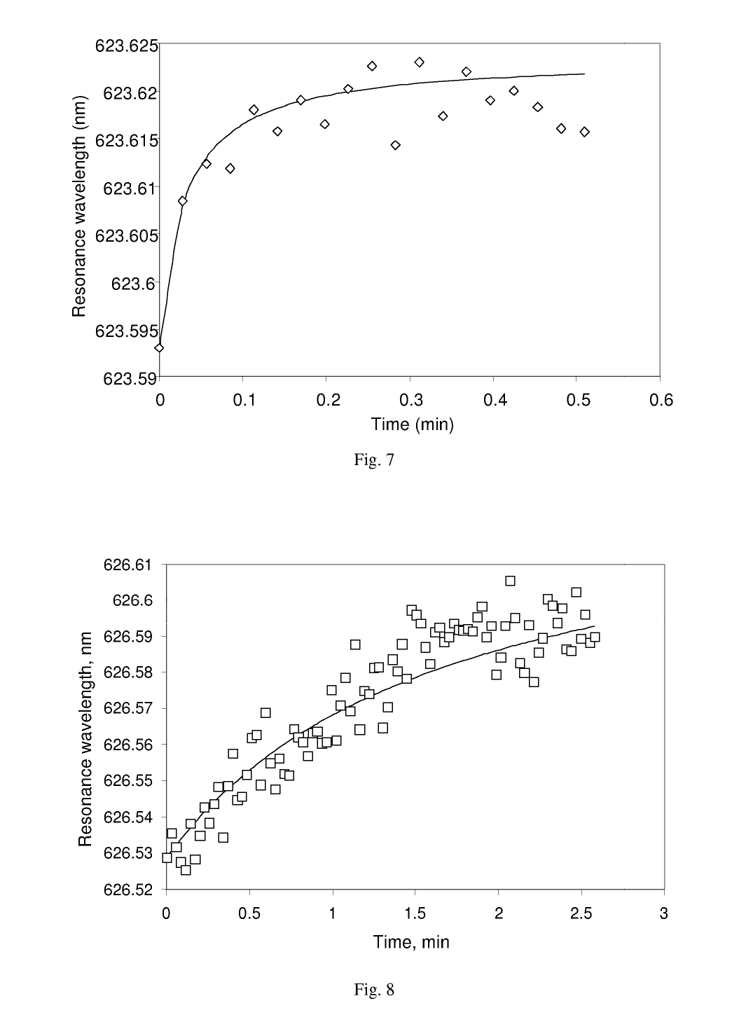

Kinetics—Layer response time was observed to vary with the type of vapor. Two typical experimental adsorption response curves are shown in FIGS. 7 and 8, where a relative saturation pressure 0.0012 benzene (FIG. 7) and 0.0012 m-xylene (FIG. 8) (i.e., partial vapor pressure was referenced to saturated vapor pressure) vapor respectively were exposed to the liquid crystal layer at room temperature. Faster response for benzene (typically t_{90%}<0.2 min) than for m-xylene (typically t_{90%}<1.5 min) was observed. This may be due to the difference in partition coefficients as a result of high difference in vapor pressure of benzene and m-xylene. However, the resonance wavelength shift at t_{90%} for benzene (0.02 nm) is less than m-xylene (0.06 nm), indicating that the change induced in the liquid crystal order by m-xylene is higher than that by benzene. This suggests that by combining analysis of both the shift in magnitude of resonance wavelength and the response time constant, selective detection may be performed.

### EXAMPLE 8

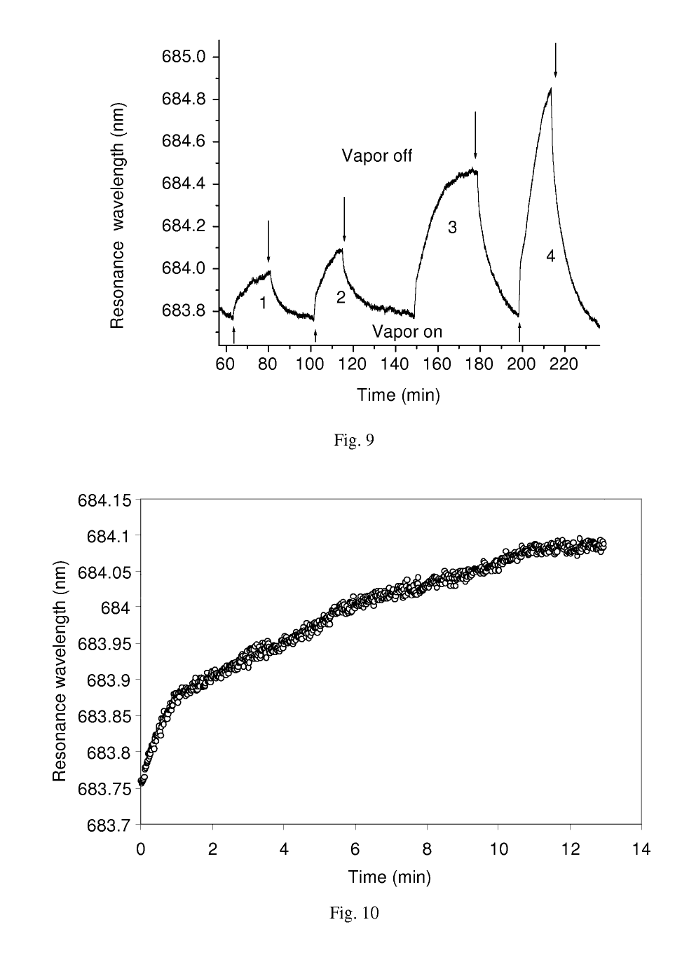

High sensitivity—To illustrate the high sensitivity that can be achieved using liquid crystals, SPR response data are shown for m-xylene vapors at 0.27 ppm to 0.85 ppm. (FIG. 9). This experiment was performed with the temperature of the liquid crystal held at about 30° C. (about 5° C. below the phase transition temperature of 5CB) whereas all the other experiments reported in this paper are at room temperature. A phase transition from ordered nematic to the disordered nematic phase was observed with as small as 5 ppm of m-xylene vapors. This is a significant reduction compared to the m-xylene concentration required to induce nematic phase transition at room temperature (780 ppm). Based on these results, the limit of detection is estimated to be about 0.02 ppm for m-xylene.

The response time may also be affected by holding the liquid crystal closer to the phase transition temperature. The adsorption kinetic (FIG. 10) has two characteristic attributes a sharp increase of response followed by a slow increase. This behavior is quite different from the kinetics observed in FIG. 7 at room temperature.

Obviously, many modifications and variations of the present invention are possible in light of the above teachings. It is therefore to be understood that the claimed invention may be practiced otherwise than as specifically described. Any reference to claim elements in the singular, e.g., using the articles “a,” “an,” “the,” or “said” is not construed as limiting the element to the singular.

### REFERENCES

All references are incorporated herein.
1. Y. Kunugi, K. Nigorikawa, Y. Harima and K. Yamashita, Chem. Soc., Chem. Commun. 873 (1994).
2. S. D. Jr. Ballantine, L. S. Rose, J. W. Grate, H. Wohltjen, Anal. Chem. 58, 3058 (1986).
3. W. J. Grate, M. H. Abraham, Review, Sens. Actuators, B Chem. 3 85 (1991).
4. B. P. J. D. Costello, P. Evans, R. J. Ewen, C. L. Honeybourne and N. M. Ratcliffe, Mater. Chem. 6, 289 (1996).
5. E. Milella, F. Musio and M. B. Alba, Thin Solid Films, 285, 908 (1996).
6. Z. K. Chen, S. C. Ng, S. F. Y. Li, L. Zhong, L. G. Xu and H. S. O. Chan, Synth. Met. 87, 201 (1997).
7. E. T. Zellers, T. S. Pan, J. S. Patrash, M. Han, S. A. Batterman, Sens. Actuators, B Chem. 12, 123 (1993).
8. R. A. McGill, M. H. Abraham, J. W. Grate, CHEMTECH. 27 (1994).
9. W. J. Grate, M. Klusty, R. A. McGill, H. M. Abraham, G. Whiting, J. A. Haftvan, Anal. Chem. 64, 610 (1992).
10. K. Buhimann, B. Schladtt, K. Cammann and A. Shulga, Sens. Actuators, B. 49, 156 (1998).
11. T. A. Dickinson, J. White, J. S. Kauer and D. R. Walt, Nature, 382, 697 (1996).
12. M. De Wit, E. Vanneste, H. J. Geise and L. J. Nagels, Sens. Actuators, B., 50, 164 (1998).
13. G. Barko and J. Hlavay, Anal. Chim. Acta, 367, 135 (1998).
14. E. Dalcanale, P. Jacopozzi, F. Ugozzoli, G. Mann, Supramol. Chem. 9, 305 (1998).
15. T. Lippmann, H. Wilde, E. Dalcanale, L. Mavilla, G. Mann, U. Heyer, S. Spera, J. Org. Chem. 60, 235 (1995).
16. Feresenbet, E.; Dalcanale, E.; Dulcey, C.; Shenoy, D. K., Sensors and Actuators B, 97, 211 (2003).
17. E. Dalcanale, P. Soncini, G. Bacchilega, F. Ugozzoli, J. Chem. Soc., Chem. Commn. 8, 500 (1989).
18. E. Dalcanale, J. Hartmann, Sensors and Actuators B, 24, 39 (1995).
19. L. W. Moore, K. N. Springer, J. X. Shi, X. Yang, B. I. Swansor, and D. Li, Adv. Mater, 7, 729(1995).
20. R. Casalini, J. N. Wilde, J. Nagel, U. Oertel, M. C. Petty, Sensors and Actuators B, 57 28 (1999).
21. A. K. Hassan, A. K. Ray, A. V. Nabok, T. Wilkop, Applied Surface Science 182,49 (2001).
22. F. L. Dickert, G. K. Zwissler, Ber. Bunsenges. Phys. Chem. 97, 184 (1993).
23. T. J. Novak, E. J. Poziomek, and R. A. Mackay, Anal. Ltrs., 5, 187 (1972).
24. E. J. Poziomek, T. J. Novak, and R. A. Mackay, Mol. Cryst Liq. Cryst. 27, 175 (1973).
25. V. K. Gupta, J. J. Skaife, T. B. Dubrovsky, and N. L. Abbott, Science.279, 2077 (1998).
26. S. J. Patrash, E. T. Zellers, Anal Chim Acta 288, 167 (1994).
27. F. L. Dickert, A. Haunschild, P. Hofmann, Anal Chem 350, 577 (1994).
28. F. L. Dickert, M. E. Zenkel, Fresenius Anal Chem, 357, 27 (1997).
29. B. Drapp, D. Pauluth, J. Krause, G. Gauglitz, Fresenius Anal Chem, 364, 121 (1999).
30. B. Kieser, D. Pauluth, G. Gauglitz, Anal Chim Acta 434, 231 (2001).
31. R. Alben, Chem. Phys., 59, 4299 (1973).
32. D. E. Martire, G. A. Oweimreen, G. I. Agren, S. G. Ryan, and H. T. Peterson, Chem. Phys. 64, 1456 (1976).
33. F. L. Dickert and K. D. Fritsch, Phys. Chem. 94, 7722 (1990).
34. B. Strehmel, J. Haferkorn, J. Stumpe, thin solid films 284, 321 (1996).
35. V. Vill, LiqCryst 2.1, Database of liquid crystals, Fujitsu Kyushu systems engineering, Fukuoka: LCI publisher, Hamburg, 1996.
36. Ru-Pin Pan, H. Hsiung, and Y. R. Shen, Phys. Rev A 36, 5505 (1987).
37. J. Homola, H. B. Lu, G. G. Nenninger, J. Dostalek, S. S. Yee, Sens. Actuators, B., 76, 403 (2001).
38. E. Kretschmann, H. Z. Raether, Naturforsch. Teil A 23, 2135 (1968).

What is claimed as new and desired to be protected by Letters Patent of the United States is:

1. A device for detecting an analyte comprising:
a substrate;
an alignment layer on the substrate;
a film comprising 4-pentyl-4′-cyanobiphenyl on the alignment layer;
a flow cell capable of delivering air suspected of containing the analyte to the film; and
an apparatus capable of measuring a physical property of the film.

2. The device of claim 1, wherein the substrate comprises a glass substrate having a chromium adhesion layer and a gold layer.

3. The device of claim 1, wherein the alignment layer comprises rubbed polyimide.

4. The device of claim 1, wherein the apparatus is capable of measuring the refractive index of the film.

5. The device of claim 1, wherein the apparatus is a surface plasmon resonance apparatus.

6. The device of claim 1, wherein the apparatus is capable of measuring a physical property of the film selected from optical, electrical, and mass.

7. The device of claim 1, wherein apparatus is selected from interferometer, capacitive transducer, atomic force microscope, surface acoustic wave device, and quartz crystal microbalance.

8. The device of claim 1, further comprising:
a system capable of correlating a change in the physical property to the concentration of the analyte.

9. The device of claim 1, further comprising:
a system capable of correlating a change in the physical property to the identification of the analyte.

10. The device of claim 1, further comprising:
a system capable of correlating the kinetics of a change in the physical property to the concentration of the analyte, identification of the analyte, or both.

11. The device of claim 1, further comprising:
a temperature controller.

12. The device of claim 1, wherein the analyte is selected from toluene, benzene, m-xylene, p-xylene, acetonitrile, and ethyl acetate.

13. A method of detecting an analyte comprising:
providing a device comprising:
a substrate;
an alignment layer on the substrate; and
a film comprising 4-pentyl-4′-cyanobiphenyl on the alignment layer;
exposing the film to air suspected of containing the analyte; and
measuring a change in a physical property of the film in response to exposing the film.

14. The method of claim 13, wherein the substrate comprises a glass substrate having a chromium adhesion layer and a gold layer.

15. The method of claim 13, wherein the alignment layer comprises rubbed polyimide.

16. The method of claim 13, wherein the physical property is the refractive index of the film.

17. The method of claim 13, wherein the measuring is performed by a surface plasmon resonance apparatus.

18. The method of claim 13, wherein the physical property is selected from optical, electrical, and mass.

19. The method of claim 13, wherein the measuring is performed by an apparatus selected from interferometer, capacitive transducer, atomic force microscope, surface acoustic wave device, and quartz crystal microbalance.

20. The method of claim 13, further comprising:
correlating the change in the physical property to the concentration of the analyte.

21. The method of claim 13, further comprising:
correlating the change in the physical property to the identification of the analyte.

22. The method of claim 13, further comprising:
measuring the kinetics of the change in the physical property; and
correlating the kinetics to the concentration of the analyte, identification of the analyte, or both.

23. The method of claim 13, further comprising:
controlling the temperature during the exposing, the measuring, or both at a temperature below the phase transition temperature of the 4-pentyl-4′-cyanobiphenyl.

24. The method of claim 13, wherein the analyte is selected from toluene, benzene, m-xylene, p-xylene, acetonitrile, and ethyl acetate.
